(19) United States
(12) Patent Application Publication
(10) Pub. No.: US 2008/0300581 A1
(43) Pub. Date: Dec. 4, 2008

(54) Device and Method for Photocoagulation of the Retina

(75) Inventors: Martin Wiechmann, Jena (DE); Manfred Dick, Gefell (DE); Diego Zimare, Pausa (DE); Regina Schuett, Jena (DE)

Correspondence Address: DARBY & DARBY P.C., P.O. BOX 770, Church Street Station, New York, NY, 10008-0770, US

(73) Assignee: Carl Zeiss Meditec AG, Jena (DE)

(21) Appl. No.: 12/094,860

(22) PCT Filed: Aug. 30, 2006
(86) PCT No.: PCT/EP2006/008493

§ 371 Date: May 23, 2008

(30) Foreign Application Priority Data
Nov. 23, 2005 (DE) 10 2005 055 885.2

(51) Int. Cl. A61B 18/20

(52) U.S. Cl. 606/4

(57) ABSTRACT

A device for photocoagulation of a retina includes a radiation source and an optical application system including a representation device configured to depict regions of the retina with sub-threshold coagulation. In addition a method for photocoagulation of the retina is provided.

This application is a U.S. National Phase application under 35 U.S.C. § 371 of International Application No. PCT/EP2006/008493, filed Aug. 30, 2006, and claims the benefit of German Patent Application No. DE 10 2005 055 885.2, filed Nov. 23, 2005. The International Application was published in German on May 31, 2007 as WO 2007/059814 under PCT Article 21 (2).

The invention relates to a device and to a method for photocoagulation of the retina.

### BACKGROUND

Light coagulation was employed for the first time at the end of the 1940s using the focused light of an axial high-pressure lamp to treat various diseases of the retina, for example, diabetic retinopathy. The retina is heated up and coagulated by the absorption of the laser beam, especially in the pigment epithelium, a dark pigmented layer located in the retina. As a result, the metabolism is focused on the regions of the retina that are still healthy. Moreover, biochemical co-factors are stimulated. As a consequence, the progression of the disease is markedly slowed or stopped.

Nowadays, lasers are usually employed as the light source. The prior-art systems for photocoagulation of the retina are based on a visual inspection of so-called coagulation foci. The radiation dose of the laser is selected at a level that is high enough that a discoloration of the retina can be visually detected. The receptors and neurofibers in the coagulation focus are destroyed in this process. A lower dose, however, is already sufficient to attain the therapeutic effect. With lower doses, a remnant of the vision could be retained—up until now, it has not been possible to create stable solutions implementing the experiments carried out with such systems that entail feedback with respect to the dose applied in order to control the so-called sub-threshold coagulation. Regions of the retina with sub-threshold coagulation cannot be opthalmoscopically detected. Regions of the retina with sub-threshold coagulation can only be rendered visible using complex methods such as, for instance, fluorescent angiography.

### SUMMARY OF THE INVENTION

An aspect of the present invention is based to provide a device and a method for photocoagulation of the retina that provides information about regions of the retina with sub-threshold coagulation and about their position.

The present invention provides a device for photocoagulation of the retina, comprising a source of radiation and an optical application system, whereby the optical application system has a representation means to depict regions of the retina with sub-threshold coagulation. Lasers are especially preferably as the source of radiation. Preference is given to the use of argon lasers, diode lasers, diode-pumped solid-state lasers, diode-pumped semiconductor lasers, YAG lasers, excimer lasers, etc. The lasers can be used in the pulsed mode or in the continuous wave (CW) mode. In addition, other light sources are also contemplated such as, for example, the focused light of a xenon lamp, light-emitting diodes (LEDs), superluminescence diodes (SLDs), etc.

Any device that can guide or aim radiation from a source of radiation is suitable as the optical application system. Such an optical application system can preferably be an optical system comprising diaphragms with appropriate profiles. Especially preferably, the optical application system can also comprise microstructured coatings on a glass substrate. The optical application system can also comprise optical fibers, controllable elements such as, for instance, small transmittive LCD panels, micro-mirror elements, diaphragms, deflection mirrors, magnifying and/or reducing optical systems, optical systems with free-form surfaces, diffractive optical systems, GRIN (gradient index) optical systems, preferably at the end of the light-conducting phase (similarly to an adapter), active elements such as, for instance, a digital mirror device (DMD), etc. With the optical application system, the beam from the source of radiation can be aimed and imaged in a predefined, spatially distributed intensity profile.

In a particularly preferred manner, the device also comprises a representation means. This representation means makes it possible to check the result of the photocoagulation, especially preferably visually. Consequently, the representation means makes it possible to check the result of the photocoagulation visually—either with the naked eye or else by means of markings that are provided. For instance, a permanent, visible change in the retina in the regions where it has been treated can serve as the representation means. This can be achieved, for example, in that a visible coagulation is brought about in the treated regions, at least partially. In this context, the organic substances present in the irradiated regions are changed in such a way that these regions can be detected opthalmoscopically. In particular, a superimposition of markings into an opthalmoscope, preferably a projection onto the retina and especially preferably a display on a monitor, is employed as the representation means.

Regions of the retina with sub-threshold coagulation are regions in which the intensity of the laser is sufficient to achieve a therapeutic effect in the pigment epithelium that is similar to the effect achieved by the visible coagulation, but not sufficient to render these regions opthalmoscopically detectable. Consequently, after the treatment, it is no longer possible to opthalmoscopically detect which regions have been treated. The retina is partially functional in the regions where sub-threshold coagulation is present.

Preferably, the representation means has a beam-modification unit with which a beam from the source of radiation can be adjusted in a predefined spatially distributed intensity profile over the surface area of the beam projected on the plane of the retina.

As a result, the large homogenous coagulation spot typical of the state of the art attains a spatially distributed intensity profile. Only in one place or in a few places is the intensity sufficient for the visible coagulation. Everywhere else, the coagulation remains in the sub-threshold range, preferably with a fixed relationship to the visually detectable region. The sub-threshold coagulation cannot be detected opthalmoscopically, the receptors and neurofibers are not destroyed or else only partially destroyed. Only in this one place or in a few places where visible coagulation is present are the receptors and neurofibers completely destroyed. These places serve for dose control.

Especially preferably, the device for photocoagulation of the retina according to the present invention comprises a beam-modification unit. Such a beam-modification unit is employed in order to adjust the beam from the source of radiation in a predefined spatially distributed intensity profile over the surface area of the beam projected on the plane of the retina. Such a beam-modification unit can comprise the above-mentioned optical elements.

The beam from the source of radiation is, for example, a light beam or laser beam that is directed out of the source of radiation by the optical application system and undergoes the requisite modifications, as a result of which the desired intensity profile can then be imaged.

Then, a predefined spatially distributed intensity profile is imaged onto the plane of the retina where the beam that has passed through the optical application system or through the beam-modification unit is supposed to act. This spatially distributed intensity profile is defined by means of the surface area of the beam projected on the plane of the retina. Therefore, the beam is applied onto the plane of the retina in a manner that is not only largely uniformly homogenous but that also entails a distribution of the intensity. Such a distribution can either be present directly statically or else it can be formed dynamically over the irradiation time.

Preferably, the spatially distributed intensity profile can be adjusted in such a way that a visible coagulation can be generated on the plane of the retina, at least in one region.

The spatially distributed intensity profile generates coagulations of varying degrees in the retina. These tend to be opthalmoscopically only partially detectable. In other words, part of the coagulation is in the sub-threshold range and part of it is visible. The regions that are opthalmoscopically detectable ideally denote the regions that cannot be detected opthalmoscopically. This is achieved, for example, in that the visibly coagulated regions form an annulus in which the regions of the retina with sub-threshold coagulation are located. Preferably, the intensity profile exhibits two different maxima. Once the region of the retina that is exposed to the highest maximum has visibly coagulated, this is preferably the signal for the surgeon that the region of the retina has coagulated sufficiently. Once the region that is exposed to the second-highest maximum has coagulated, this is preferably a signal for the surgeon to discontinue the irradiation of the retina as soon as possible. Preferably, the intensity distribution can be adjusted. Especially preferably, the ratio of the various intensities of the intensity profile are variable with respect to each other. Particularly preferably, the ratio of the beam intensities that are supposed to bring about a visible or sub-threshold coagulation can be adjusted. As a result, the intensity profile can be adapted to various retinas in such a way that during surgery, the regions that are intended for sub-threshold coagulation are irradiated at an optimal intensity. As a result of the fact that the intensity of the beam that is to bring about a visible coagulation can be adjusted independently, the duration of the irradiation that the surgeon will select can be optimally adjusted. Therefore, the sub-threshold coagulation can be reliably reproduced.

A visible coagulation can be opthalmoscopically detected. The receptors and neurofibers are destroyed in the regions of the retina where a visible coagulation is present. A therapeutic effect is achieved. Without any additional auxiliary means, the surgeon can opthalmoscopically detect which regions have been treated. However, the functionality of the retina is destroyed in visibly coagulated regions.

In a preferred embodiment of the present invention, the intensity profile encompasses one or more defined maxima which, in total, comprise a surface area of less than 20%, preferably less than 10%, especially preferably less than 5% of the surface area encompassed by the surface area of the beam projected on the plane of the retina.

Especially preferably, the intensity profile thus encompasses defined maxima that have a greater intensity than the rest of the area covered by the beam from the source of radiation. In this context, the surface area that is occupied by the maxima relative to the total irradiated surface area is less than 20%, preferably less than 10% and especially preferably less than 5%. In this manner, only a small part of the irradiated retina is injured in order to provide a visual confirmation of the coagulation, while the remaining region only displays sub-threshold coagulation and thus retains a certain amount of vision. Therefore, by restricting the surface areas irradiated with the maxima, the portion of the retina within the irradiated surface area that is not completely destroyed by the coagulation can be pre-determined. If the intensity profile encompasses several maxima, the surface area consists of the total of the appertaining maxima.

Through the selection of more than one maximum, it is preferably possible to indicate the mid-point of the beam area of the specific corner points of the irradiated surface area that is directed onto the retina—in this manner, the region of the retina that has already been irradiated can then be visually checked. Thus, for instance, four maxima can be represented at the same distance on the outer edge of the irradiated surface area configured as an annulus, thus depicting the irradiation in this specific region.

In another preferred embodiment of the present invention, the intensity of the at least one maximum can be adjusted at a fixed ratio with respect to the intensity of the remaining area of the intensity profile.

As a result of this largely fixed ratio of the intensity with respect to the maximum of the remaining area of the intensity profile, a defined relationship is preferably predefined between the degree of coagulation that is achieved among the regions irradiated with the maxima and the remaining area. In this manner, it is possible to apply a uniform pre-specified dose of radiation to the retina, thus bringing about a sub-threshold coagulation. The visible coagulation points that now appear and that were caused by the maxima thus serve to confirm that a pre-specified dose uniformly acted upon the rest of the irradiated surface area.

In another preferred embodiment of the present invention, the intensity of the maxima is sufficient for the visible coagulation, while the intensity of the remaining area of the intensity profile is less than 80%, preferably less than 60%, especially preferably less than 50%, of the intensity of the maxima.

The intensity of the maxima is preferably selected in such a way that it suffices for the visual inspection of the coagulation, while the intensity of the remaining area of the intensity profile is irradiated less intensely. Owing to this difference in the radiation intensity, the irradiation can be visually checked or detected, whereby a fixed relationship relative to the radiation intensity exists for the area outside of the maximum. Consequently, with irradiation at a pre-selected ratio between the maximum and the rest, it can be assumed that a specific (not directly verifiable visually) radiation dose has been used when the visible coagulation of the remaining region has been reached. This ratio can also be individually adapted to the circumstances of the patient in question, so that a ratio is taken for a given patient that differs from that which is needed for another patient. This ratio can be ascertained experimentally in a preliminary examination. Especially preferably, this is done in a calibration mode before the actual treatment. The ratios thus obtained between the maximum on the one hand and the dose of the irradiation of the remaining surface area on the other hand is then preferably retained in a patient-specific manner. It is particularly preferred for such a calibration to take place in a region of the retina that is not very decisive for the actual vision.

In another preferred embodiment of the present invention, several maxima have predefined intensities that differ from each other.

Owing to the formation of several maxima having different predefined radiation intensities, the irradiation can be adapted even more precisely. For instance, by selecting three maxima, a treatment can be configured in such a manner that the irradiation is terminated after the visible appearance of two maxima—therefore, the occurrence of one maximum serves as an indication for the surgeon that the dose can still be increased, whereas the presence of three maxima tells the surgeon that the treatment should be terminated at this point in time at the latest. The selection of appropriate gradations between the maxima can thus provide an additional optical aid for the continuation of the irradiation of the region in question.

In another preferred embodiment of the present invention, the intensity profile can be generated statically or dynamically.

On the one hand, an intensity profile can be generated statically or dynamically. A static realization of the intensity profile can be done, for example, by means of appropriate optical systems, lens systems or free-form surfaces, via which the intensity of the beam is kept constant over the entire time of the treatment. This can also be a series of very short pulses whose intensity profile is formed by the appropriate optical systems.

It is likewise contemplated to generate the intensity profile dynamically. The dynamic generation of an intensity profile can be achieved, for example, through a course over time of the intensity of the beam, so that when the intensity rises, a higher dose and thus a corresponding profile can be applied in specific regions of the surface area of the retina onto which the beam has been projected. Thus, it is also possible, for example, to employ scanners or diaphragms, diffractive optical systems or digital mirror devices to change the intensity curve of the irradiation over time so that only at pre-specified regions is a higher intensity profile applied than in the other regions.

In another preferred embodiment of the present invention, the beam-modification unit comprises a diaphragm having a defined profile.

The appropriate intensity profile can be specified by means of a diaphragm with a defined profile by activating the diaphragm or by means of partial absorption of the beam within the diaphragm. Particularly preferred in this context are microstructured coatings on, for instance, a glass substrate. Such coatings make it possible to generate specific intensity profiles through the absorption of the beam or by masking off partial beams.

In another preferred embodiment of the present invention, the maxima can be adjusted along a concentric ring around the mid-point of the surface area of the beam projected on the plane of the retina.

Owing to the spatial configuration of the maxima on the projected surface area, figures can be displayed that are easy to recognize geometrically and that, during the visual inspection, not only depict when the visible coagulation has been achieved but also at the same time mark the region where the non-visible irradiation of the remaining region has taken place. Thus, for instance, circular segments or a full circle can be used to represent the region where coagulation has taken place. By the same token, by having various maxima on a circle, the total area that has been irradiated can be marked with dots. For instance, by marking three maxima on the circumference of a circle, it can already be reliably indicated in which (remaining) area irradiation has taken place.

Especially preferably, a concentric ring around the mid-point of the irradiated surface area is selected. In addition, it is also possible to realize wedge-shaped figures.

In another preferred embodiment of the present invention, the maxima can be generated in a calibration mode so as to be variable over time.

Especially preferably, by varying the intensity of the radiation in a calibration mode, it can be ascertained prior to the actual treatment at what power density the coagulation threshold will be exceeded. The subsequent coagulations to treat the rest of the retina are then carried out within the sub-threshold range with a homogenous spot or irradiated surface area. Thus, only in the calibration mode, for example, a wedge-shaped intensity attenuator is swiveled into the optical path. This attenuator can preferably be in the form of a grey wedge, a dielectric graduated coating, a micro-optically diffractive or refractive element or else by means of active elements such as digital mirror devices (DMD), etc. In the device according to the invention, this calibration step can preferably be repeated at different places. Particularly preferably, the calibration step is always carried out at the beginning of a treatment and if necessary repeated in the intervening time, for example, in the case of regions of the retina that absorb in a significantly different manner. Especially preferably, the calibration is performed in regions of the retina that are functionally less important, while the purely sub-threshold coagulation treatment is done in functionally important regions of the retina.

This device according to the invention allows a retina treatment with the reassurance of a calibration, which also allows the surgeon to select the degree of the sub-threshold coagulation by adjusting the power. Thanks to this capability of a calibration of the device according to the invention, a coagulation device is provided that offers an especially gentle irradiation and treatment of the retina.

Preferably, the device comprises a representation means with which at least one marking can be depicted. Regions with sub-threshold coagulation are not visible to the surgeon. Once they are provided with a marking, the positions of these regions can be displayed to the surgeon. Therefore, regions of the retina can be marked in such a way that the markings assist the surgeon by serving as a reminder during the surgery.

When the device according to the invention is used for photocoagulation of the retina in such a way that the coagulation only suffices for visible coagulation at one place or at a few places of the coagulation spot, the representation means which is able to depict at least one marking can be used for the additional representation of coagulation spots. Consequently, either during or after the treatment, a surgeon can better detect opthalmoscopically which places of the retina have been coagulated. Therefore, during the treatment, the surgeon does not lose track of the treated sites of the retina. Otherwise, there would be the risk that the surgeon might treat individual sites several times or else that regions that needed to be treated are left untreated. If the treatment takes place in several sessions or if different surgeons perform the surgery, it is better if the surgeon can keep track of the treated sites. This eliminates the need for the surgeon to remember the treated sites and to write them down on a form after the surgery.

Preferably, computer animation is employed as the representation means. Markings at a specific distance from each other can be displayed in computer animation. In this context, 3D-computer graphics or 2D-computer graphics can be used. The 2D-computer graphics are preferably generated in the form of vector graphics. These consist of geometrical shapes and can thus be scaled as desired. The 2D-computer graphics can also be in the form of raster graphics. Raster graphics consist of dots that can be scaled although this results in quality losses. More complex images can be described even better with raster graphics.

Preferably, the positions of the laser spots that cause sub-threshold coagulation can be detected by a camera during the coagulation, then fed to a computer that records the image of the coagulated retina at the point in time of the laser actuation, together with an image of the retina obtained prior to this, where the position of the laser coagulation and the diameter of the spot are stored. This position can either be displayed on a separate monitor, superimposed into the application system or projected onto the retina.

Preference is also given to feeding the coordinates of the laser scanner to a computer that determines the position of the laser coagulation on this basis.

Preferably, the representation means comprises an output device. Devices that are suitable for depicting markings can be used as the output device. The markings can be output temporarily or permanently. Preferably, the output device is configured to depict markings in various colors. Especially preferably are output devices that can represent markings three-dimensionally. Likewise especially preferably are output devices that can show markings in animation. Preferably a monitor, especially a color monitor, is used as the output device. Examples of monitors are cathode-ray tube monitors, liquid-crystal monitors or plasma monitors. Devices that generate holograms, printers or plotters can also be employed as the output devices.

As the output device, preferably an opthalmoscope is employed into which markings are superimposed. The markings can be superimposed into a direct opthalmoscope as well as into an indirect opthalmoscope. A direct opthalmoscope contains an illumination system, an observation system and correction lenses, thus being configured in such a way that an examiner can observe the patient's eye directly, without an intermediate image being generated.

Preferably, the markings are superimposed into an indirect opthalmoscope. In the case of an indirect opthalmoscope, an intermediate image is generated that is observed by the examiner. Here, the retina is observed using a light source that is directed at the patient's eye at a distance of about 50 cm, and a magnifying glass that is held at a distance of about 2 cm to 10 cm from the patient's eye.

Preferably, the markings are projected onto the retina. A surgeon can thus opthalmoscopically observe the markings together with the retina.

Preferably, the markings are easy to recognize. For instance, light points can be employed as the markings. Markings of different shapes, different colors, three-dimensional, blinking or animated markings can all be used.

A three-dimensional display of the markings is preferably achieved by displaying two half-images or an image pair in a stereoscopic arrangement or with stereoscopic image information. For the sake of simplicity, only the term “half-images” will be employed below. This, however, also refers to an image pair in a stereoscopic arrangement or with stereoscopic image information. Each of the two half-images is made accessible to one eye. This can be done by superimposing the half-images into the corresponding optical paths of a slit lamp or opthalmoscope, or else by observing the half-images with an auxiliary means that makes each of the two half-images accessible to a given eye. The auxiliary means can be in the form of, for instance, color filters, polarizing filters or this can be achieved by alternately covering one eye. When the markings are projected onto the retina, preferably the focal position of the projected markings are adapted to the curvature of the retina.

The region of the retina that has been treated with the beam from the source of radiation can be identified preferably by means of a marking of a first type. This makes it easy for the surgeon to keep track of the treated regions.

A marking of the type described above can be used as the marking of a first type.

The representation means is preferably configured to apply a marking of a second type onto regions of the retina that are to be treated with the beam. Consequently, the surgeon can easily keep track of the regions of the retina for which a treatment is planned.

A marking of the type described above can be likewise used as the marking of a second type. If markings of different types are used at the same time, the markings preferably differ from each other considerably. This can be achieved, for example, by selecting different colors, shapes, a different size or geometry or else visible features that change over time. It is likewise possible for a marking of the second type to be shown as a blinking marking and, after the treatment, to then be shown as a steady marking of the first type. By the same token, a marking of the second type can be shown rotated once the appertaining region of the retina has been treated.

In particular, it is preferred if the representation means is configured to place a background image behind the marking. As a result, the position of the marking can be unambiguously ascertained.

The background image should facilitate the orientation of the surgeon on the basis of the markings. This is preferably done by using a clearly structured background image by means of which the background is divided into individual areas, or else by means of a representation of a retina. For instance, a photograph, a graph, a film or an animation can be employed for this purpose. Preferably, the background image allows the markings to stand out clearly. In order to do so, the background image and the markings can be provided, for example, in complementary colors. Preferably, various background images are shown alternately behind the markings.

It is particularly preferred to employ a coordinate system as the background image. By doing this, the position of the individual markings can be unambiguously determined in a simple manner.

For example, a Cartesian coordinate system or a polar coordinate system can be selected as the coordinate system.

Especially preferably is the use of a fundus image as the background image. This makes it particularly easy for the surgeon to mentally transfer the markings to the real fundus image in front of him.

An image of the retina of the patient to be treated is preferably employed as the fundus image. However, an image of another retina could also be used. In this manner, the surgeon could compare the retina of the patient being treated to another retina. Preferably, the images of different retinas are shown consecutively.

Especially preferably, the background image is three-dimensional. This makes it possible to adapt the background image to the retina. The position of the markings can thus be rendered very accurately. It is the very easy for the surgeon to mentally transfer the markings to the reality.

A three-dimensional background image is an image that additionally provides the observer with depth information for each point of the image. Preferably, a three-dimensional background image consists of two half-images that can be observed directly or by means of suitable auxiliary means in such a way that each one is perceived by only one eye. A preferred possibility for observing the image with auxiliary means consists of coloring the two photographs differently and observing them with color filter eyeglasses. In this context, the colors and color filters are selected in such a way that each time, one half-image can be viewed through a color filter. Another possibility to make a given half-image visible to each eye is to employ the polarization filter technique. Here, projectors are preferably used to project the two half-images onto the same place. Polarized filter films rotated by 90° are positioned in front of the projection objectives. The observer views the projected image through polarized filter eyeglasses that have been appropriately provided with polarized filter films. Preferably, 3D images are observed with shutter eyeglasses. For this purpose, a monitor, for instance, alternately shows the image for the left eye and for the right eye. The shutter eyeglasses correspondingly covers the left and the right eyes alternately.

Especially preferably, the background image is a live image. In this manner, the surgeon can observe the changes that occur during the surgery together with the markings.

Preferably, the current image of the retina of the patient is shown as the live image.

Preferably, the representation means is configured to display the number of the regions of the retina that have been treated with the beam from the source of radiation. As a result, the surgeon can quickly gain an impression of how many regions of the retina he has treated.

This number is preferably shown in one corner. This hardly interferes with the depiction of the markings.

This number is preferably shown as a digit or as a countdown. The number is shown in ascending order. But it is likewise possible to display the number of planned treatment regions at the beginning of the surgery and for this number to be counted down during surgery.

Here, it is practical if the representation means is configured in such a way that the markings and their completion are displayed online. In this manner, the surgeon can see the current status at all times during surgery.

In this context, information that a marking is to be placed is sent directly to the representation means during the treatment of the retina.

In order to determine the regions that are to be marked, for instance, a computer can receive the information that a beam has been aimed at the retina. Together with this information, the starting site and the direction of the beam could be indicated. On the basis of these three pieces of information, the computer can determine the point where a coagulation point is located in the retina. Subsequently, the computer can prompt the representation means to show a marking there.

The regions to be marked can also be determined by a camera that records the retina during the treatment and by a computer that detects the treated regions on the basis of the images taken. The camera could be fastened, for example, to a laser slit lamp or to a slit lamp with a laser link.

Here, it is particularly preferred if the device is provided with a memory to store the markings, the fundus image and/or the coordinate system. Thus, the markings can be superimposed in the case of a subsequent treatment.

Semiconductor memories such as a flash memory, magnetic memories such as hard drives or optical memories such as CDs can all be employed as the storage medium.

The present invention also provides a method for photocoagulation of the retina, whereby a representation means represents regions of the retina with sub-threshold coagulation.

Preferably, the representation means comprises a beam-modification unit with which a beam from the source of radiation can be aimed with a distributed intensity profile at the retina, as a result of which a visually detectable coagulation can be detected only in areas of a maximum of the intensity profile.

Preferably, the representation means marks different regions of the retina with sub-threshold coagulation.

Preferably, the representation means has a camera with which the regions of the retina that have been treated with the beam from the source of radiation can be detected. In this manner, the treated regions can easily be detected.

As the camera, preference is given to the use of a device that can detect images as still images or animated images.

The camera is preferably a photo camera. The photo camera preferably takes a picture of the retina at the point in time when the retina is being treated with the beam from the source of radiation. As a result, exactly the information that is of interest to the surgeon is detected in each case. Examples of photo cameras that can be used are digital cameras or analog cameras. The use of a digital camera has the advantage that the images can immediately be further processed by a computer. The use of an analog camera has the advantage that the images can be acquired very accurately on a photo film.

Especially preferably, the camera is a film camera. Using the film camera, the retina is preferably filmed from the beginning to the end of the surgery. This even more reliably ensures that an image exists of all of the treatments that the retina underwent during surgery. Preferably, an electronic camera is employed as the camera. As a result, the acquired images are of high quality. Special preference is given to the use of a video camera as the camera. This translates into a cost-effective acquisition of the images.

Preferably, the representation means comprises a computer with which the regions of the retina that have been treated with the beam from the source of radiation can be marked on a coordinate system or on a fundus image. The information that a given region of the retina has been treated with the beam from the source of radiation, for example, in the form of an image or by an indication of the coordinates, can be entered into a computer. This information can then be processed in the computer and subsequently output. For purposes of the output, the computer can mark the areas in a coordinate system or onto a fundus image. Preferably an electronic circuit, especially preferably a computer, is used as the computing means.

The computer is preferably configured in such a way that markings can be superimposed into the observation optical path of a surgeon. Thus, the markings are shown to the surgeon in a very convenient manner. The surgeon's observation optical path into which the markings are superimposed is preferably located in a slit lamp, especially preferably in an opthalmoscope. It is very easy to superimpose a marking into a slit lamp. The superimposition into an opthalmoscope is particularly practical since opthalmoscopes are normally employed in laser surgery.

The invention will now be illustrated on the basis of figures depicting other advantageous embodiments. The figures show the following:

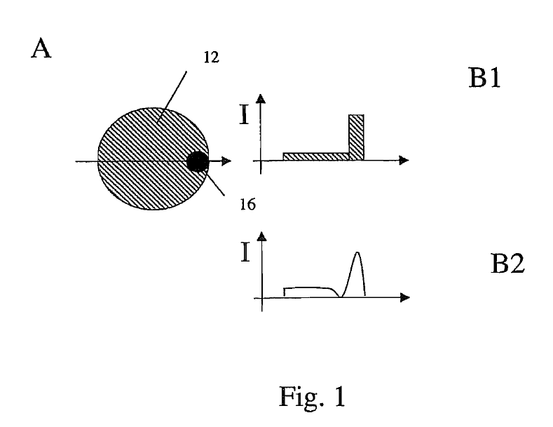

FIG. 1—a schematic top view of a projected surface area and graphs of the intensity distribution;

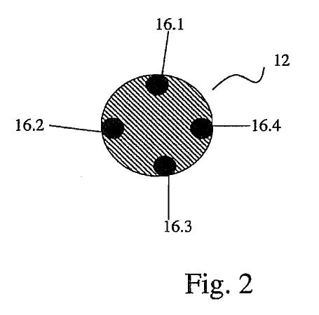

FIG. 2—an embodiment of an intensity profile according to the invention on a projected surface area;

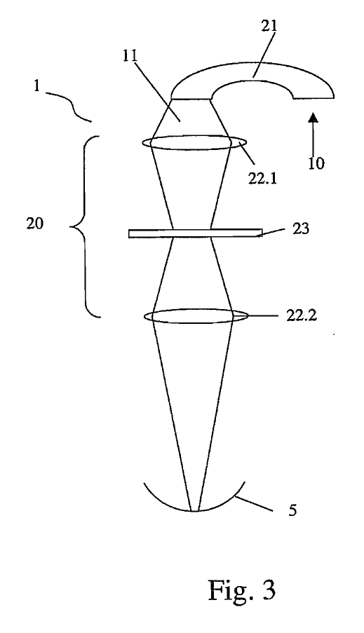

FIG. 3—a schematic depiction of an embodiment of a device according to the invention for photocoagulation;

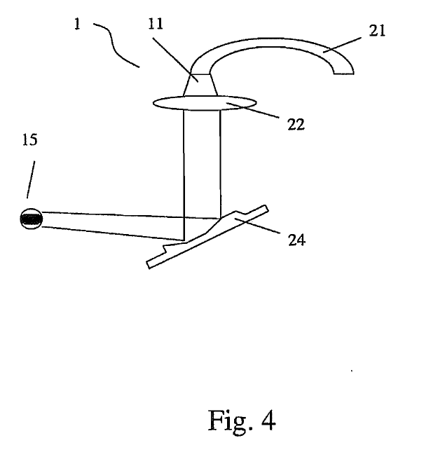

FIG. 4—another embodiment of the device according to the invention for photocoagulation;

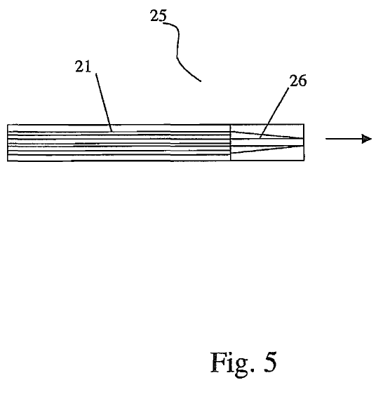

FIG. 5—another embodiment of the beam-modification unit according to the invention;

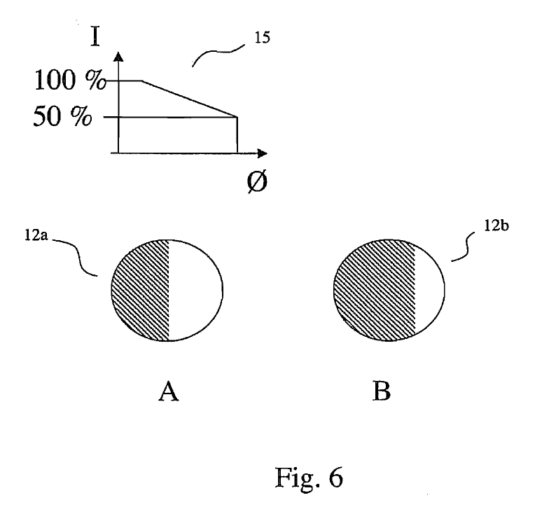

FIG. 6—a schematic depiction of a diagram with the intensity profile and two examples of projected surface areas;

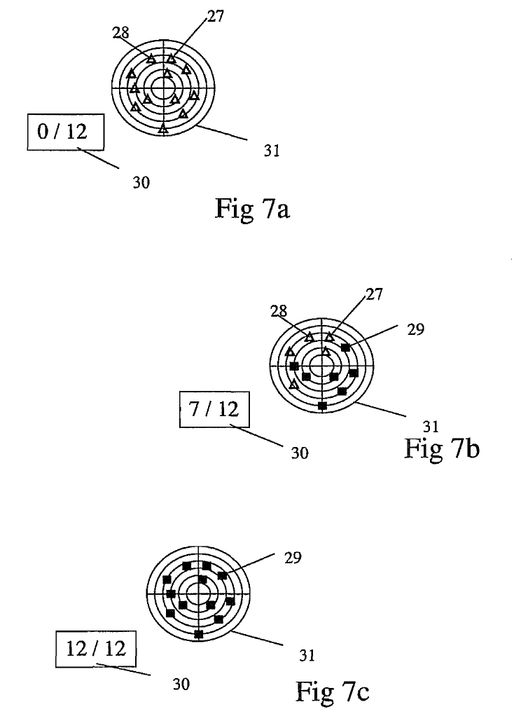

FIG. 7—a schematic depiction of markings of a first and second type; and

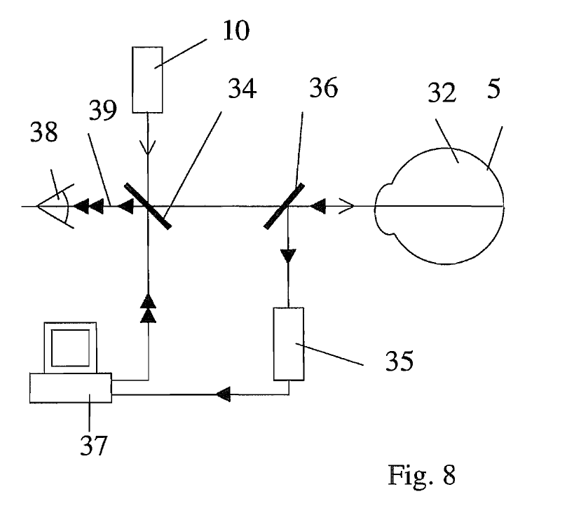

FIG. 8—a schematic depiction of an embodiment of a device according to the invention for photocoagulation.

### DETAILED DESCRIPTION

In FIG. 1, Diagram A shows a projected surface area 12 which encompasses an intensity maximum 16. The homogeneous intensity profile of the laser spot thus exhibits a region of higher intensity 16 that can be visually recognized during the coagulation. Diagram A depicts the intensity distribution in a top view onto the projected surface area 12 of the plane of a retina. The dark maximum 16 indicates a high radiation intensity.

Diagram B1 depicts the intensity profile along a section through the spot shown in Diagram A along the indicated center line. In this cross section, the intensity is low and constant over a large surface area and increases in the region of the maximum 16. Diagram B1 depicts an ideal intensity distribution as it should be represented on the retina.

In reality, owing to thermal conduction in the retina, it could be advisable to calculate an intensity distribution that differs from this ideal case, which then results in an intensity distribution on the retina after the thermal compensation effects. In other words, for example, it might be necessary to place an appropriate maximum next to a minimum that remains below the desired radiation intensity since thermal compensation effects are also taken into consideration that continue to have an effect stemming from the intended maximum.

The retina is only destroyed at the site of the maximum 16. This site serves for dose control. In the remaining region of the irradiated surface area 12, the coagulation remains at the sub-threshold level, that is to say, the function of the retina is largely retained.

In FIG. 2, a projected surface area 12 is shown on which several intensity maxima 16.1 to 16.4 are imaged. The intensity maxima 16.1 to 16.4 are arranged along the circumference of the circular projected surface area 12. These maxima 16.1 to 16.4 not only indicate that the radiation intensity has been reached but also mark the place where the projected surface area was applied. Here, too, the surface area with sub-threshold coagulation predominates (crosshatched), while the individual maxima 16i [sic] only occupy a small part of the surface area. It is also possible to select three maxima which, arranged on the circular plane, likewise depict the region of the projected surface area 12. It is likewise contemplated that a maximum is arranged in the form of a ring and largely contiguous, whereby the ring is preferably arranged around the mid-point of the projected surface area 12. Therefore, the four points shown in FIG. 2 could depict such a ring it they were to be connected with each other on a circle. In this manner, the actual location of the coagulation can be clearly seen after the treatment, even though the sub-threshold coagulation cannot be rendered visible. This simplifies any subsequent treatment and localization of places already coagulated. Preferably, the maximum can thus also be implemented as a ring or in the form of a donut profile. This translates into better localization, especially in the case of smaller spots.

FIG. 3 shows a schematic set-up of a device for photocoagulation 1. The device for photocoagulation 1 comprises an optical fiber conductor 21 as well as an optical application system 20. The optical application system 20 consists of a first lens 22.1, a diaphragm 23 and a second lens 22.2

A source of radiation 10 is coupled to the optical fiber conductor 21 and emits a beam 11. This beam 11 is guided through the optical application system 20 and projected through the first lens 22.1 onto the diaphragm 23. The beam 11 passes through the latter and is focused onto the retina 5 by the second lens 22.2. As a result, in the vicinity of an intermediate plane of the laser beamed in the laser zoom onto the retina, an appropriate profile is made on the diaphragm 23 which, in this case, encompasses a microstructured coating on a glass substrate. The beam is shaped in accordance with the profile prescribed here or else imparted with an appropriate intensity profile. Preferably, the diaphragm 23 is exchangeable so that profiles with differing shapes and transmission courses can be prescribed. The diaphragm 23 can also encompass controllable elements such as small transmittive LCD panels that provide a high degree of flexibility in terms of the shape and intensity ratios. In this context, the intermediate image plane can preferably be expanded once again so that the LCD panels are not destroyed by the laser intensity. By the same token, it is also possible to employ micro-mirror elements such as, for instance, digital mirror devices (DMD). Here, the optical beam path is preferably folded open since these elements work on the basis of reflection. The optical application system 20 here is preferably configured as a zoom system. In this manner, the beam 11 is applied onto the retina 5 by the profile imparted by means of the diaphragm 23 with the appropriate intensity distribution.

FIG. 4 shows another embodiment of the device according to the invention for photocoagulation. Here, an optical fiber conductor 21 is provided by means of which the beam 11 is rectified by a lens 22 and deflected onto a free-form surface area 24 that is configured as a deflection mirror. Therefore, a corresponding profile is predefined on the free-form surface, said profile having the now deflected beam 11 and thus resulting in an intensity profile 15 on the retina 5, as is indicated by way of an example with the reference numeral 15 in FIG. 4. Thus, owing to the optical system having free-form surfaces, another possibility is created to generate different intensity profiles. The deflection mirror could also be configured so that it can be switched over. A magnifying and reducing optical system located downstream could continuously vary the scale and thus switch the profile on and off. By means of this method, it would be likewise possible to generate a delineation that diverges from the round shape.

FIG. 5 shows another embodiment of a possible beam-modification unit 25. The end of an optical fiber conductor 21 has a GRIN optical system 26 configured as an adapter. GRIN is the abbreviation of “graded/index” or “gradient/index”. In this optical element, the refractive index is location-dependent. With a GRIN lens, the refractive index changes continuously as a function of the path in the medium. Thus, in the GRIN optical system 26 in FIG. 5, two small maxima are formed that, in a sectional view, are arranged around the mid-point of the surface area being irradiated. The intensity distribution is thus transformed into the desired intensity distribution by the GRIN optical system 26 at the end of the fiber. This intensity distribution can then be further imaged by the prior-art optical system and thus be transferred to the retina 5.

FIG. 6 shows an embodiment in which, in an initial calibration step, a wedge-shaped intensity course over the beam cross section is applied onto the retina. The diagram depicts the intensity distribution and this wedge-shaped intensity course that, over the diameter of the applied spot, decreases from a 100% intensity to a 50% intensity. Diagrams A and B then show the projected surface areas 12a and 12b that constitute two different results on two different retinas. It can be seen in Diagram A that the coagulated region that can be detected visually makes up approximately 50% of the surface area. This area is crosshatched and can be seen on the left-hand side of Diagram A. The right-hand side is not detectably coagulated. Taking into consideration that the intensity on the left side of the intensity profile that acts on the projected surface area 12a fell from 100% to 50% on the right-hand side, it can be concluded that the visible coagulation occurred at intensities of more than 75% of the pre-selected intensity. Now, in order to select a value at which no visible coagulation occurs, the surgeon will want to choose a value of less than 75%.

In the other irradiated projected surface area 12b, approximately 80% has coagulated along the wedge-shaped intensity distribution. Consequently, only 20% is not coagulated. Therefore, the surgeon can read off that, in this case, he can only select an intensity of less than 60% of his wedge-shaped intensity course from 100% to 50% so that no visible coagulation occurs.

Due to this wedge-shaped intensity course over the cross section of the beam that is applied onto the retina, it can be detected in a patient-specific manner at which power density the coagulation threshold is exceeded. The subsequent coagulations are then carried out in the sub-threshold range with a homogeneous spot. Only in the calibration mode is a wedge-shaped intensity attenuator swiveled into the optical path. This can be, for instance, a grey wedge, a dielectric graduated coating, a micro-optically diffractive or refractive element or else by means of active elements such as digital mirror devices (DMD). In this context, the calibration step is preferably carried out at the beginning of a treatment and, if necessary, it can be repeated in the intervening time, for example, in the case of regions of the retina that absorb in a significantly different manner. Preferably, the calibration is performed in regions of the retina that are functionally less important, while the purely sub-threshold treatment is preferably done in functionally important regions of the retina.

The advantage of this embodiment of the invention is thus the therapeutically effective retina treatment with sub-threshold coagulation, along with the reassurance of a preceding calibration and also the fact that the surgeon can select the degree of application of the sub-threshold coagulation on the basis of the pre-selected adjustment of the power.

Therefore, the solution being presented here provides a device and a method with which the retina coagulation can be performed in a gentle manner so that, through the treatment and the appertaining feedback provided by the visually detectable coagulation centers, the retina can largely retain its function in the irradiated regions.

FIG. 7 shows a schematic depiction of markings of a first and a second type. Here, the markings of a first type denote regions of the retina that have been treated with a laser. The markings of a second type denote regions of the retina that are intended for a treatment. These markings are shown to the surgeon through an opthalmoscope during surgery.

The depiction of FIG. 7a shows an image that is displayed to a surgeon at the beginning of the surgery. The depiction in FIG. 7b corresponds to an image that is displayed during the surgery. The depiction in FIG. 7c corresponds to an image that is displayed to the surgeon at the end of the surgery.

FIG. 7a shows a polar coordinate system 31. This polar coordinate system 31 designates several regions of the retina of a patient who is to be treated. Twelve triangles are distributed on the polar coordinate system as markings of a second type. Only the contours of the triangles are shown here. Through the opthalmoscope, the surgeon would see them as solid red triangles. Two adjacent numbers are shown to the left of the coordinate system. The figure on the left stands for the number of treated regions and the one on the right for the total number of regions to be treated. Here, the figure shown on the left is “0” and to the right of it the figure “12”. Since FIG. 7a shows the image at the beginning of the surgery, no treated regions are shown here yet, but rather only twelve regions that are intended for treatment.

Diverging from FIG. 7a, FIG. 7b shows only five red triangles. In the places in the coordinate system where the remaining triangles are depicted in FIG. 7a, there are now solid black squares as markings of the second type. The solid black squares appear in green through the opthalmoscope. The numbers “7” and “12” are depicted to the left of the coordinate system. In the state shown in FIG. 7b, 7 out of 12 regions intended for treatment have been treated.

Diverging from FIG. 7b, FIG. 7c shows solid black squares at the places where the triangles can be seen in FIG. 7b. Moreover, the number “12” appears twice next to the coordinate system. In the state shown in FIG. 7c, all 12 regions intended for treatment have been treated.

Every time, one of the triangles shown in FIGS. 7a and 7b is displayed as a blinking marking to the surgeon. The blinking triangle denotes a region of the retina that is intended as the one to be treated next within the scope of a pre-planned treatment sequence.

The display of these markings allows the surgeon to clearly see during the surgery which regions have already been treated. He can also see which regions are still to be treated and which region is intended as the next for treatment. The display of the numbers to the left of the polar coordinate system allows the surgeon to quickly obtain an overview of the progress of the surgery. The colors red and green have been chosen for the two markings since they are easy to distinguish from each other. The red triangle, which denotes the region that is intended as the one to be treated next, blinks because this makes it particularly conspicuous. Moreover, this also provides the opportunity to make it clear that the region thus marked belongs to the group of regions that have not yet been treated in order to nevertheless emphasize it. The color black has been chosen for the polar coordinate system 30 since it is so visible but at the same time it does not distract the attention of the surgeon away from the red triangles 27 and the green squares 28.

FIG. 8 shows a schematic depiction of an embodiment of a device according to the invention for photocoagulation. The eye of a patient 32 is shown on the right-hand side. By means of an observation optical path 39, one eye of a surgeon or of a treating physician 38 is directed towards the retina 5 of the eye 32. A laser 10 is aimed at the retina 5 via a first deflection unit 34. A camera 35 is aimed at the retina 5 via a second deflection unit 36. Here, the camera 35 is installed in a laser slit lamp or a slit lamp with a laser link (not shown here). The camera 35 is connected to a computer 37. The computer 37 has a connection to the observation optical path 39.

During the surgery or treatment, the retina 5 is observed by the camera 35 via the second deflection unit 36. This provides a live image of the retina. The live image is relayed to the computer 37. On the basis of this information, the computer 37 automatically detects the position of the treated regions of the retina at the point in time of the irradiation with the laser 10 or of the laser shot. The detected position of the treatment point or the site of treatment is then marked or drawn into a coordinate system or into a standard orientation system or into a fundus image of the patient. In this embodiment, this is done in that the standard orientation system for the retina is superimposed live with a fundus image of the patient.

The marking or the dot or the drawing is superimposed or mirrored into the observation optical path 39 of the treating physician 38 by means of the computer 37. In addition, the coordinate system or the standard orientation system is superimposed into the observation optical path 39. Moreover, the currently actuated shot number 30 and additional treatment parameters are superimposed into a corner of the field of vision.

In the treatment being presented here, regions already treated are read into the computer 37 and superimposed with the live fundus image or with a new fundus image. Regions of the retina 5 that are intended for treatment or laser foci that have been planned prior to the treatment on the basis of a fundus image are superimposed into the optical path during the surgery or treatment. During subsequent treatments, additional treatment points are superimposed into the fundus image or into the image. For purposes of differentiating the markings of various treatments and for the planning of the treatment, the markings are color-coded and have different shapes. Markings for the planning of the treatment are shown as red crosses. The regions treated during the ongoing treatment are marked with green circles. Regions that were treated during previous surgeries are marked with black dots.

After the treatment, the markings or the recorded treatment pattern or the treatment sites are stored in a patient database by the computer 37.

As a result of the fact that the treated sites are stored, they can be called up and superimposed once again during a subsequent treatment. The surgeon can thus obtain an overview of all of the treatments performed on an eye 32. Consequently, a different surgeon can take over the surgery without any problems.

As a result of the fact that a marked fundus image or a marked standard orientation system accompanies the current fundus image, the surgeon is constantly receiving feedback about the position of the treated regions or the positions of laser shots that have already been carried out. Therefore, at all times, the surgeon has a current overview of the regions already treated.

Recording the treated regions with a camera 35 is an efficient and cost-effective way to record these treated regions. Besides, the acquired data can be easily relayed to a computer 37.

The superimposition of the markings into the observation optical path 39 of the eye of a surgeon 38 is particularly convenient for the surgeon. While he is looking at the retina 5, he can, at the same time, see the markings.

1-28. (canceled)

29: A device for photocoagulation of a retina comprising:
a radiation source; and
an optical application system including a representation device configured to depict regions of the retina with sub-threshold coagulation.

30: The device as recited in claim 29, wherein the representation device includes a beam-modification unit configured to adjust a beam from the radiation source in a predefined spatially distributed intensity profile over the surface area of the beam projected on a plane of the retina.

31: The device as recited in claim 30, wherein the spatially distributed intensity profile is adjustable so that a visible coagulation can be generated in at least one region of the plane of the retina.

32: The device as recited in claim 30, wherein the distributed intensity profile encompasses at least one defined maxima, which, in total, comprises a surface area that is less than 20% of the surface area of the beam projected on the plane of the retina.

33: The device as recited in claim 32, wherein an intensity of the at least one maxima is adjustable at a fixed ratio with respect to an intensity of the remaining area of the intensity profile.

34: The device as recited in claim 33, wherein the intensity of the at least one maxima is sufficient for a visible coagulation, while the intensity of the remaining area of the intensity profile is less than 80% of the intensity of the maxima.

35: The device as recited in claim 32, the at least one defined maxima includes a plurality of maxima, each maximum having a predefined intensity that differ from the other maxima.

36: The device as recited in claim 30, wherein the intensity profile can be generated statically or dynamically.

37: The device as recited in claim 30, wherein the beam-modification unit includes a diaphragm having a defined profile.

38: The device as recited in claim 32, wherein the at least one maxima is adjustable along a concentric ring around a mid-point of the surface area of the beam projected on the plane of the retina.

39: The device as recited in claim 32, wherein the at least one maxima can be generated in a calibration mode so as to be variable over time.

40: The device as recited in claim 29, wherein the representation device is configured to depict at least one marking.

41: The device as recited in claim 40, wherein the at least one marking includes a first marking for identifying a region of the retina that has been treated with the beam from the radiation source.

42: The device as recited in claim 40, wherein the at least one marking includes a second marking on regions of the retina that are to be treated with the beam.

43: The device as recited in claim 40, wherein the representation device is configured to place a background image behind the at least one marking.

44: The device as recited in claim 43, wherein the background image includes a coordinate system.

45: The device as recited in claim 43, wherein the background image includes a fundus image.

46: The device as recited in claim 43, wherein the background image is three-dimensional.

47: The device as recited in claim 43, wherein the background image is a live image.

48: The device as recited in claim 29, wherein the representation device is configured to display a number of the regions of the retina that have been treated with the beam from the radiation source.

49: The device as recited in claim 29, wherein the representation device is configured to display markings online.

50: The device as recited in claim 29, further comprising a memory to store at least one of markings, a fundus image and a coordinate system.

51: The device as recited in claim 29, wherein the representation device includes a camera configured to detect regions of the retina that have been treated with the beam from the radiation source.

52: The device as recited in claim 29, wherein the representation device includes a computer configured to mark, on one of a coordinate system and a fundus image, regions of the retina that have been treated with the beam from the radiation source.

53: The device as recited in claim 52, wherein the computer is configured so that markings can be superimposed into an observation optical path of a surgeon.

54: A method for photocoagulation of the retina, the method comprising:
directing a beam from a radiation source at the retina; and
representing regions of the retina with sub-threshold coagulation using a representation device.

55: The method as recited in claim 54, further comprising marking the regions of the retina with sub-threshold coagulation using the representation device.

56: A method for photocoagulation of the retina, the method comprising:
directing a beam from a radiation source with a distributed intensity profile at the retina so that a visually detectable coagulation can be detected only in areas of a maximum of the intensity profile.
